(19) United States
(12) Patent Application Publication
(10) Pub. No.: US 2006/0271197 A1
(43) Pub. Date: Nov. 30, 2006

(54) Spinal disc annulus augmentation

(76) Inventors: Jeffrey Alan Saal, Portola Valley, CA (US); Joel Stuart Saal, Portola Valley, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/442,641

(22) Filed: May 26, 2006

Related U.S. Application Data
(63) Continuation-in-part of application No. 11/139,731, filed on May 26, 2005.

(51) Int. Cl. A61F 2/44

(52) U.S. Cl. 623/17.16

(57) ABSTRACT

Intervertebral disc implants are provided for augmenting the annulus of the disc in a manner to bear at least part of the axial and/or torsional load on the annulus so that rents, fissures and subsequent herniation of the disc are prevented or substantially delayed. An aspect of the subject devices is that they have an operative height dimension that is equal to or less than the disc height of a normally functioning, healthy disc. Methods and tools are also provided for the minimally invasive implantation of the device within an intervertebral disc.

### CROSS-REFERENCE TO RELATED APPLICATIONS

This application is a continuation-in-part of U.S. patent application Ser. No. 11/139,731, filed on May 26, 2005, incorporated herein by reference.

### FIELD OF THE INVENTION

The present invention is directed towards the augmentation of the annulus of intervertebral discs.

### BACKGROUND OF THE INVENTION

The spinal column is formed from a number of bony vertebral bodies separated by intervertebral discs which primarily serve as a mechanical cushion between the vertebral bones, permitting controlled motions (flexion, extension, lateral bending and axial rotation) within vertebral segments. The normal, natural intervertebral disc is comprised of three component tissues: the nucleus pulposus (“nucleus”), the annulus fibrosis (“annulus”), and two opposing vertebral end plates. The two vertebral end plates are each composed of thin cartilage overlying a thin layer of hard, cortical bone which attaches to the spongy, richly vascular, cancellous bone of the vertebral body. The nucleus is constituted of a gel-like substance having a high (about 85%) water content.

The annulus is an outer fibrous ring of collagen fibers that surrounds the nucleus and binds together adjacent vertebrae. The fibers of the annulus consist of 15 to 20 overlapping multiple plies, called lamellae, and are inserted into the superior and inferior vertebral bodies at roughly a 30-degree angle in both directions. As about half of the angulated fibers will tighten when the vertebrae rotate in either direction, this configuration helps to resist twisting or torsional motion. The annulus is generally about 10 to 15 millimeters in height and about 15 to 20 millimeters in thickness, occupying about ⅔ of the intervertebral space.

With aging and continued stressing, the nucleus becomes dehydrated and/or one or more rents or fissures may form in the annulus of the disc. Such fissures may progress to larger tears which allow the gelatinous material of the nucleus to migrate out of the nucleus and into the outer aspects of the annulus which may cause a localized bulge. In the event of annulus rupture, the nuclear material may escape, causing chemical irritation and inflammation of the nerve roots.

Posterior protrusions of intervertebral discs are particularly problematic since the nerve roots are posteriorly positioned relative to the intervertebral discs. Impingement or irritation of the nerve roots not only results in pain in the region of the back adjacent the disc, but may also cause radicular pain such as sciatica. Nerve compression and inflammation may also lead to numbness, weakness, and in late stages, paralysis and muscle atrophy, and/or bladder and bowel incontinence.

Progressive degeneration of the disc also leads to a reduction in disc height thereby increasing the load on the facet joints. This can result in deterioration of facet cartilage and ultimately osteoarthritis and pain in the facet joints.

A common treatment for a disc protrusion is discetomy, a procedure wherein the protruding portion of the disc is surgically removed and the weakened or ruptured portion of the annulus is repaired Discectomy procedures have an inherent risk since the portion of the disc to be removed is immediately adjacent the nerve root and any damage to the nerve root is clearly undesirable. Furthermore, discectomy procedures are not always successful in the long term as the annulus fibrosis has been shown to have limited healing capacity increasing the risk of re-herniation. A compromised annulus may lead to accelerated disc degeneration which may require spinal interbody fusion or total disc replacement.

Accordingly, it would be highly advantageous to be able to treat or inhibit intervertebral disc degeneration, particularly degeneration of the annulus fibrosis, at early stages of degeneration so as to obviate or postpone the need for discectomy, fusion, partial or total disc replacement and/or other surgical options. It would be additionally advantageous to provide techniques and procedures for augmenting the annulus which are minimally invasive, requiring minimal disruption of the annulus.

### SUMMARY OF THE INVENTION

The present invention provides devices for augmenting the intervertebral disc annulus. The devices are particularly useful in treating, delaying or preventing the consequences and problems associated of degeneration of the disc. The invention further includes methods directed to the minimally invasive implantation of one or more devices of the present invention within an intervertebral disc annulus.

These and other features, objects and advantages of the invention will become apparent to those persons skilled in the art upon reading the details of the invention as more fully described below.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIG. 1A shows a sagital cross-section of a functional spine unit;

FIG. 1B shows a top axial view of a portion of the inferior vertebrae and the intervertebral disc of the functional spine unit of FIG. 1A;

FIG. 2A shows a sagital cross-section of a functional spine unit having an annulus which has been augmented by the implantation of a device of the present invention;

FIG. 2B shows a top axial view of the inferior vertebrae and the intervertebral disc of the functional spine unit of FIG. 2A having a subject device operatively implanted therein;

FIG. 2C shows an enlarged view of a section of the annulus of the functional spine unit of FIG. 2B in which the subject device is implanted.

FIGS. 3A-3C show side, end and top views, respectively, of an embodiment of the augmentation device of the present invention.

FIG. 4 shows a top view of another embodiment of an augmentation device of the present invention.

FIGS. 5A and 5B show top and side views, respectively, of another embodiment of the augmentation device of the present invention.

FIGS. 6A and 6B show side and end views, respectively, of another embodiment of the augmentation device of the present invention.

FIGS. 7A and 7B show side and end views, respectively, of another embodiment of the augmentation device of the present invention.

FIGS. 8A and 8B show side and end views, respectively, of another embodiment of the augmentation device of the present invention.

FIGS. 9A and 9B show side and end views, respectively, of another embodiment of the augmentation device of the present invention.

FIG. 10 shows a side view of another embodiment of an augmentation device of the present invention.

FIGS. 11A and 11B show side and end views, respectively, of another embodiment of the augmentation device of the present invention.

FIGS. 12A-12D illustrate various steps for implanting a device of the present invention.

FIG. 13 illustrates another embodiment of a pair of augmentation devices of the present invention operatively implanted within a spinal disc.

FIG. 14A illustrates another embodiment of a pair of augmentation devices of the present invention operatively implanted within a spinal disc; and FIG. 14B illustrates the device having anchor members in undeployed and deployed configurations.

FIG. 15 illustrates another embodiment of a pair of augmentation devices of the present invention operatively implanted within a spinal disc.

FIG. 16A illustrates two other embodiments of the augmentation device of the present invention operatively implanted within a spinal disc; FIG. 16B illustrates one of the devices prior to insertion; and FIG. 16C illustrates the other device having anchor members in undeployed and deployed configurations.

FIG. 17 illustrates another embodiment of the augmentation device of the present invention operatively implanted within a spinal disc.

FIG. 18 illustrates another embodiment of a pair of augmentation devices of the present invention operatively implanted within a spinal disc.

### DETAILED DESCRIPTION OF THE INVENTION

Before the subject devices, systems and methods are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. For example, in this description and the following claims, the terms “anterior”, “posterior”, “superior” and “inferior” are defined by their standard usage in anatomy, i.e., anterior is a direction toward the front (ventral) side of the body or functional spine unit; posterior is a direction toward the back (dorsal) side of the body or functional spin unit; superior is upward toward the head; and inferior is lower or toward the feet.

As used herein, the term “intra-annular space” includes any portion of the annulus between the inner aspect and the outer aspect of the annulus, including but not limited inter-lamellar space, i.e., between two adjacent lamellae, wherein the position, extent or thickness of the inner aspect of the annulus may be dynamic as the disc degenerates and/or as a person ages, i.e., the inner aspect of the annulus tends to blend deeper into the nuclear space with increasing degeneration. As such, the term “intra-annular space” also includes “potential” annular space.

The present invention will now be described in greater detail by way of the following description of exemplary embodiments and variations of the devices and methods of the present invention. The invention generally includes an implantable annulus augmentation device or apparatus as well as instruments and methods for the percutaneous or minimally invasive implantation of the device. The devices are particularly suited for treating earlier-stage disc degeneration.(i.e., grades I-IV) but may also be used to treat later stage degeneration requiring fusion, disc replacement or other surgical interventions.

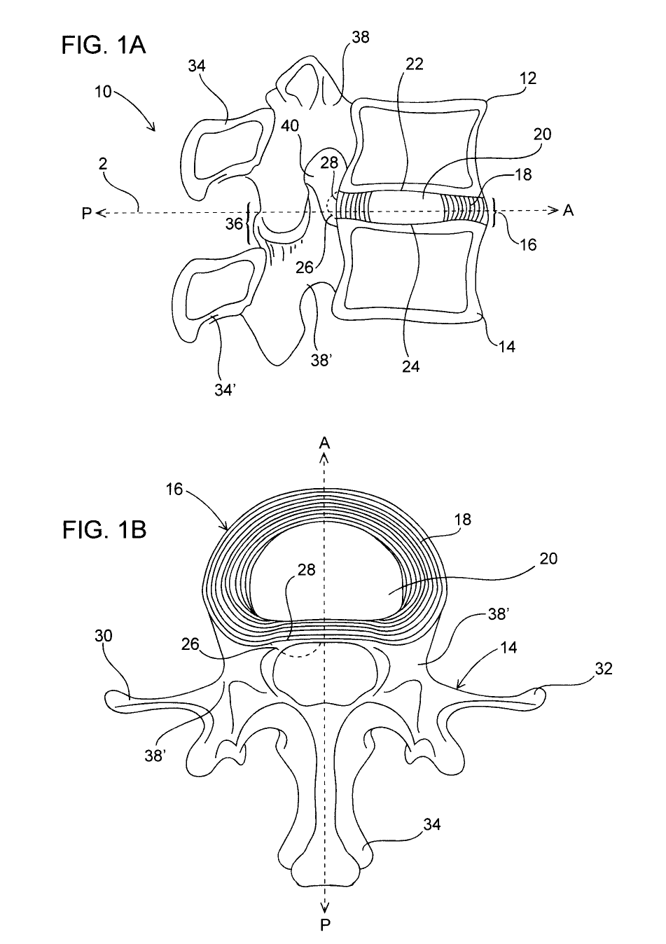

Referring now to FIGS. 1A and 1B, the general anatomy of a functional spine unit 10 is illustrated. Axis 2 shows the anterior (A) and posterior (P) orientation of the functional spine unit within the anatomy. A functional spine unit includes the bony structures of two adjacent vertebrae (superior vertebral body 12 and inferior vertebral body 14), the intervertebral disc 16 (including the annulus fibrosis 18, the nucleus pulposus 20, and endplates 22, 24 of the vertebrae), and the ligaments, musculature and connective tissue (not shown) connected to the vertebrae. Intervertebral disc 16 substantially fills the space between the two vertebral bodies to support and cushion them, and permits movement of the two vertebral bodies with respect to each other and other adjacent functional spine units. Extending posteriorly from each of vertebral bodies 12 and 14 are left and right transverse spinous processes 30, 32 and a posterior spinous process 34, 34′. The vertebral bodies also include facet joints 36 and pedicles 38, 38′ that form the neural foramen 40.

As discussed above, progressive degeneration of the disc results in disc height loss where the superior vertebral body 12 moves inferiorly relative to the inferior vertebral body 14. Loss of disc height may result in buckling of the annular fibers which leads to deterioration of the annulus along with fissures and tears within the annulus, and ultimately herniation of the disc, as illustrated by herniated segment 26, shown in phantom, which protrudes beyond the posterior border 28 of annulus 18.

The present invention is directed to augmenting the annulus in a manner to bear at least part of the axial, torsional and/or shear loads on the annulus so that rents, fissures and subsequent herniation of the disc, and the pain resulting therefrom, are able to be prevented or substantially delayed, or if already existing, then repaired or at least stabilized against further deterioration. This is accomplished by implantation of one or more augmentation devices within the annulus, and particularly between adjacent lamellae or plies of the annulus; however, the implanted device need not reside completely within the annulus and may, in part, reside within the nucleus.

The construct and material characteristics of the subject devices are such that they mimic the physical properties of a natural, healthy annulus. In other words, the subject devices provide physiological abilities and limits corresponding to those of the natural annulus so as to enable natural motion of the spinal motion segment in each rotational and translational direction (i.e., flexion, extension and lateral bending) and to support normal physiological loads (i.e., axial loading) exerted on the spine. In addition to having properties to enable normal spinal motion, the subject devices have characteristics (e.g., stiffness) to resist bending, fatigue failure, displacement, deformation, torsional stress, shear loads and other repetitive stresses and strains undergone by the disc, and particularly the annulus, during normal spinal motion.

Another aspect of the subject devices is that they have an operative thickness dimension which is less than an operative height dimension. By “operative” it is meant that the dimensions are those which exist upon or after complete deployment of the device at the implant site, i.e., subsequent to achieving a fully expanded condition. In general, the ratio of the operative thickness to the operative height of the subject devices is typically about 1:15 (thinnest) to about 1:3 (thickest), but the ratio may be higher or lower depending on the application. For example, where a defect within the annulus involves several layers of lamellae, it may be desirable to use a thicker device. Most commonly, the thickness-to-height ratio is in the range from about 1:10 to about 1:7.

Another aspect of the subject devices is that they have an operative height dimension that is less than or equal to the disc height of a normally functioning, healthy disc when unloaded. As such, when implanted, a subject device may be positioned within the annulus such that it engages both, one or neither of the endplates of the adjacent vertebrae between which it has been implanted. With devices having a height that is substantially equal to (or greater than if such is desired) than the axial dimension of the normal disc space, the devices further function to buttress or shore up the annulus at least at the site of implant within the annulus which in turn increases the stability of the intervertebral disc. Depending upon what grade of disc degeneration exists at the time of implantation, the implanted device functions either to maintain disc height, or to increase the disc height from an initial height that is less than optimal to an acceptable height and subsequently maintain the increased height.

While certain embodiments of the subject devices have a fixed configuration, shape and/or dimensions, in order to enable the most minimally invasive implant procedure possible, the devices may have or be put in a lower profile or configuration during delivery, and subsequently transitioned to their operative size and/or height, as will be described in greater detail below.

To achieve the objectives of the present invention, the thickness of a subject augmentation device when in a deployed or expanded or high profile condition may range from about 0.5 mm to about 5 mm, and most typically from about 1 mm to about 3 mm, and the height in a deployed, expanded or high profile condition may range from about 3 mm to about 15 mm, and most typically from about 4 mm to about 10 mm. In their undeployed, unexpanded or low profile condition, the subject devices may have a thickness as narrow as about 0.5 mm, and a height as short as about 2 mm. Accordingly, the thickness dimension of a device in a high profile state may be as much as about three times the thickness of the device when in the low profile condition, the height dimension of a device in a high profile state may be as much as about twice the height of the device when in the low profile condition.

The devices may have any practical or suitable length where the length of a particular device is usually no greater than the circumference of the lamellar plane in which the device is implanted. Longer device segments may be preferred if, in addition to torsionally and axially supporting the disc, it is desirable to exert radial or circumferential compression on the disc in order to close rents and fissures already present within the annulus. Typically, the length of a single device may substantially traverse the entire circumference of an intra-annular layer but is typically in the range from about 15 mm to about 100 mm, and most typically from about 20 mm to about 50 mm, which dimensions may be less when the device is in an undeployed, unexpanded or low profile condition.

In certain embodiments, the thickness of the implant may vary along its length.

For example, where an implant is used to address points of weakness, e.g., rents, spaced apart from each other in the same intra-annular space or plane, the portions of the length of the device which are to be positioned at the targeted points may have a thickness greater than the intermediate and distal portions of the device. Alternatively or additionally, one or both ends of the device may have a thickness which varies from the remainder of the device length. For example, the distal end, i.e., the end of the device that leads during delivery and implantation of the device, may be thinner so as to be more flexible and thus more easily steered and manipulated between the targeted intra-annular layers. Conversely, it may be desirable for the proximal end of the device to have a thickness greater than the remainder of the device length, in order to be more pushable through a catheter and between the lamellae.

The length of the device is typically, but not required to be, greater than the height of the device, and as such, has an elongated configuration. Depending on the length of the device, it may be straight (shorter device segments) or have a radius of curvature along its length (longer device segments) which matches that of the intra-annular circumference.

The augmentation devices may have any suitable shape given the above dimensions. Typical configurations include but are not limited to flat planar strips or ribbons, or coils, tubes, cylinders or arcs having a straight, circular, semi-circular, elliptical, etc. cross-sectional shape. Particularly with those embodiments which are intended to reside at least partially in the nucleus, the device may have a U shape or overlapping configuration where the end portions of the device extend substantially parallel to each other from a bend or curve at a more central portion along the device length. Particularly because a portion of the device traverses one or more lamellar layers in a crosswise direction, such a configuration is inherently self-retaining.

In certain embodiments, the devices may have reinforcing members such as struts, braces or the like to further fortify the device against forces exerted on it within the disc. Still yet, the devices may be provided with anchors affixed on the top and/or bottom edges for anchoring into the endplates of the vertebral bodies. Alternatively or additionally, anchors may be provided on the side edges of the devices and/or along their lamellar contacting surfaces for anchoring into the lamellae. In other embodiments, particularly with those intended to partially reside within the nucleus, the end of the device configured to be positioned within the nucleus may have an anchoring structure which may be transitionable from a low-profile or undeployed state to facilitate delivery into the disc and to and expanded or deployed state to brace or anchor the device within the disc. For the latter embodiments, suitable materials include shape memory materials which transitions from one profile to the other by a change (e.g., increase) in temperature. The temperature change may be passive, such as that experienced by the device upon implantation into the body, or may be active where energy is applied to the device structure which causes it to achieve a preformed shape. Alternatively, the device may be made of a superelastic material whereby the anchoring end is constrained in the undeployed state until ready for deployment, upon which the restraint (e.g., delivery tube/catheter) is removed to deploy the anchor members.

Certain embodiments are made of a malleable material that allows the device to be bent or conformed to a desired configuration either before or during implant. Particularly with lengthier devices, e.g., those used that are intended to traverse a substantial circumference of a lamellar layer or are intended to be positioned in both a portion of the annulus and nucleus, a device which is malleable along its length can be customized for a disc's particular condition(s). Also, it allows for versatility in the manner in which the device is stabilized within the disc. For example, one or both ends of the device may be navigated to and positioned between the annulus and the nucleus and/or extending into the nucleus, within the transition zone between the nucleus and annulus, or into the nucleus from the annulus and back into the annulus where the ends ultimately reside completely within the annulus and a portion therebetween resides within the nucleus.

As mentioned above, the entirety of the device (or a substantial portion thereof), rather than just an anchoring end or member thereof, may be expansile, i.e., have an unexpanded or undeployed state in which they are delivered to the intra-annular implant site and then expanded or deployed upon proper positioning at the implant site. The transition from an undeployed state to a deployed state may require an active step such as pulling a wire or filament attached to the implant. Alternatively, the device may be configured to be self-expanding or self-deploying whereby its release from a delivery sheath is sufficient to expand or deploy the device. Still yet, the device may be made of a hydrophilic material which is able to absorb intra-annular fluid or be injected with a fluid, thereby expanding to an optimal size or height until absorption has reached equilibrium.

Other embodiments included in the present invention include balloons which are expandable or inflatable with a biocompatible material. The balloon material is preferably non-compliant or semi-compliant so as to maintain a substantially fixed shape or configuration and ensure proper, long-term retention within the implant site. The material may be non-porous material to prevent seeping of the inflation or expansion medium from the balloon. Suitable materials include polyurethane, silicone, or polycarbonate-polyurethane. The inflation or expansion medium may be saline or another biologically compatible fluid, or a flowable solid material, such as polyurethane, or a gel, which thickens or hardens substantially upon injection into the balloon. The injectable material may be a curable material such as silicone or polyurethane. The filler material may be curable by chemical reaction (e.g., moisture), photo-activation (e.g., UV light) or the like. The cure time is preferably sufficiently long to enable activation just prior to insertion (i.e., outside the body) and permit sufficient time for navigation and positioning of the augmentation device in the disc. However, activation may also take place inside the body after implantation. The balloon may have an inflation or injection port at a proximal end for coupling to a source of inflation or expansion material or medium. The port may consist of a one-way valve which is self-sealing upon release from an inflation or expansion mechanism or tube.

The augmentation devices may be made of a biocompatible material or coated with a biocompatible material. The devices may be constructed of inert materials, such as metals and polymers, or of biologic materials, such as autologous or cadaver tissue, or heterologous or organic material. Examples of suitable metals include stainless steel and super elastic alloys such as nickel titanium (Nitinol). Examples of suitable polymers include LPLA (poly(1-lactide)), DLPLA (poly(dl-lactide)), LPLA-DLPLA, PGA (polyglycolide), PGA-LPLA, PGA-DLPLA and the like. An example of a biologic material that may be used is collagen or cellulose. Other metals, alloys, polymers, and composites having suitable tensile, compression and fatigue strength and elasticity may also be used. For balloon-type embodiments, a non-porous material such as latex or acrylate may be used.

The devices may incorporate chemicals, e.g., to balance the ph of the surrounding tissues or to increase oxygenation of the tissue; cellular elements including but not limited to stem cell lines, activated macrophages, monocytes and/or other immuno-active cellular elements; drugs such as anti-inflammatory agents and analgesics; vitamins, anesthetics, neurotropics, neuromodulators, co-factors or trace elements that will enhance tissue viability; and/or other biological agents such as growth factors, anti-cytokine factors, etc. to facilitate healing of the damaged portions of the annulus. The devices may be further constructed to have the ability to elute any of the above in a time-release fashion.

A single implant may be used at a discrete point of weakness, e.g., a rent, in the annulus or a longer implant may be used to address multiple points of weakness about the circumference of the annulus. Multiple devices may be implanted within the same intra-annular plane, i.e., between the same two adjacent lamellae, or may be implanted within different intra-annular planes. Where more than one device are implanted, they may be positioned serially along a circumference of the annulus or in parallel to provide a stacked arrangement. With the stacked arrangement, the devices may be placed such that they are separated from each other by at least one lamella or two or more may be placed between the same adjacent lamellae where at least a portion of their lengths overlap each other.

Various exemplary embodiments of annulus augmentation devices are now described; however, such description is not intended to be limiting but exemplary of the present invention. Any combination of features, materials, functions and physical characteristics described above may be applied to each of the devices of the present invention.

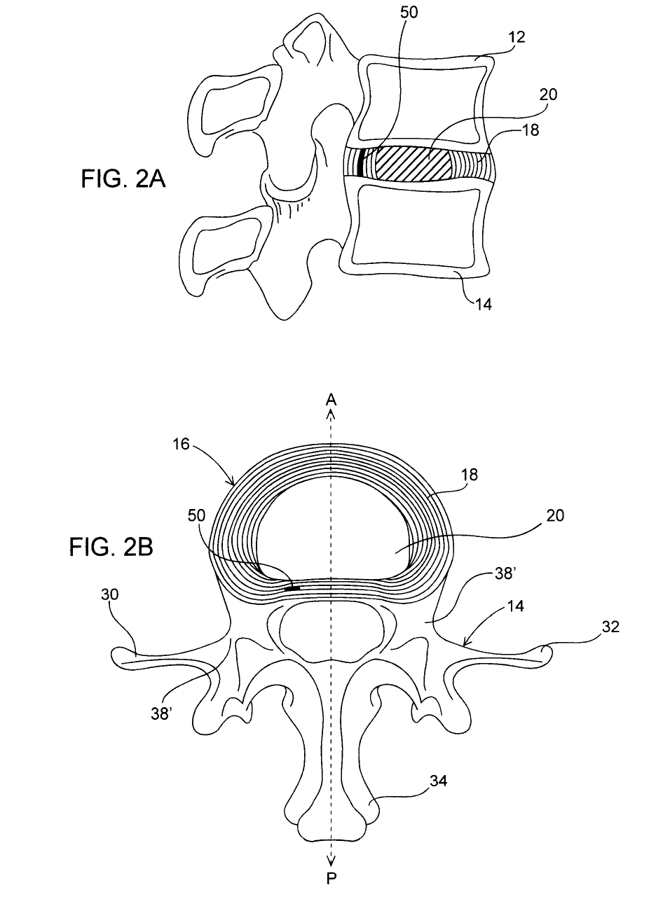

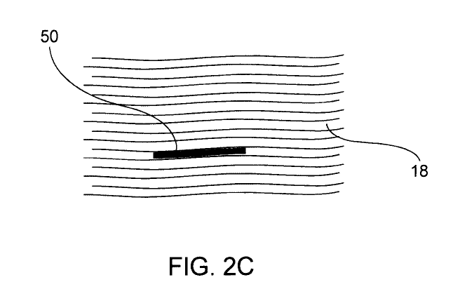

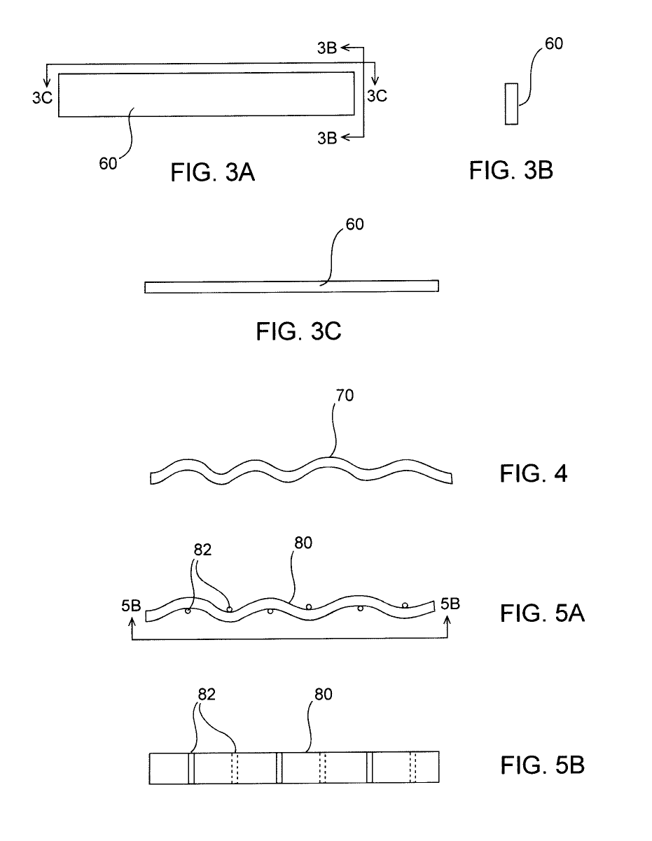

As mentioned above, certain variations of the devices are configured to be implanted completely within the annulus and others are intended to be implanted partially within the annulus. FIGS. 2A-2C illustrate a schematic representation of the former variation in which a subject device 50 is implanted wholly within the annulus. More specifically, device 50 is positioned between adjacent lamellae of the posterior portion of annulus 18. FIGS. 3-12 illustrate various exemplary embodiments of devices which are suitable for such application.

Referring now to FIGS. 3A-3C, side, end and top views, respectively, of an augmentation device 60 of the present invention having a straight, flat strip or ribbon configuration. FIG. 4 illustrates a top view of another device 70 having a similar ribbon configuration but is wavy or rippled along its length. FIGS. 5A and 5B are top and side views, respectively, of device 80 having a plurality of struts 82 space apart both sides of the ribbon.

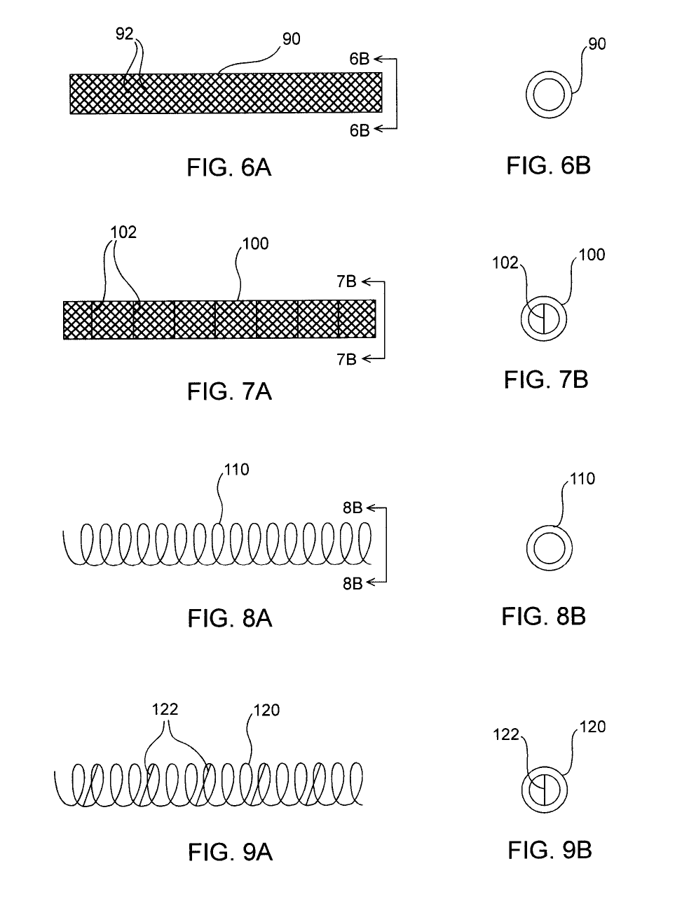

FIGS. 6A and 6B illustrate side and end views, respectively, of augmentation device 90 formed from cross-woven filaments 92 to provide an expandable mesh or cage having a tubular configuration. The mesh or cage maybe made of a super-elastic memory material which is compressible for delivery through a cannula and which is self-expanding upon implantation. FIGS. 7A and 7B illustrate side and end views, respectively, of a similar tubular device 100 having a plurality of struts 102 vertically positioned within the lumen of device 100. Upon expansion, the mesh tube 100 may be self-retaining whereby its struts 102 are sufficiently rigid by themselves to maintain the expanded condition and withstand the natural forces exerted on it by spine.

FIGS. 8A and 8B illustrate side and end views, respectively, of augmentation device 110 formed from a spring or coil configuration. FIGS. 9A and 9B illustrate side and end views, respectively, of a similar coiled device 120 having a plurality of struts 122 vertically positioned within the lumen of device 120.

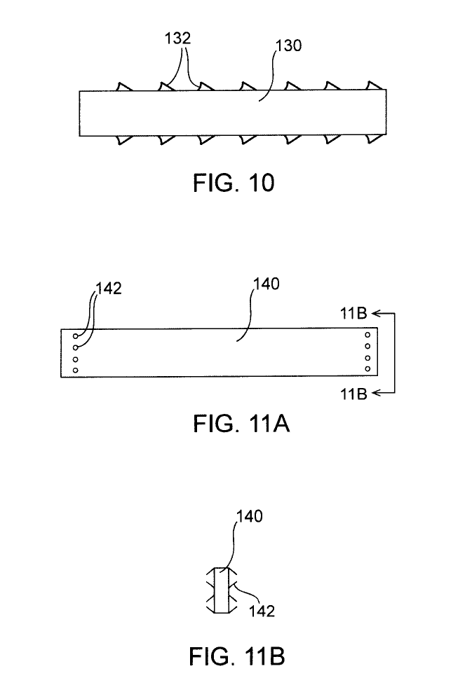

As mentioned previously, the implantable devices of the present invention may be provided with anchors for better securing the device at the implant site and to prevent migration of the device therefrom. For example, FIG. 10 illustrates a ribbon device 130 having anchors in the form of keels 132 extending from the top and bottom edges of the devices. The keels are designed to penetrate into the endplates of the vertebral bodies between which the device is implanted. Keels 132 may be angled so as to facilitate forward advancement of device 130 within the intervertebral disc. FIGS. 11A and 11B illustrate a ribbon device 140 having anchors 142 at proximal and distal ends of device 140 in the form of pins or spikes which penetrate into the annulus lamellae between which device 140 is sandwiched. The anchors may be fixed or movable so as to be retracted or flush with the device surface during delivery and subsequently deployed upon final placement of the device within the annulus.

As mentioned above, a feature of certain embodiments of the augmentation devices of the present is that they are expandable or deployable from a low profile configuration to a higher profile or operative configuration. This design allows the device, when in the low profile condition, such as a narrow, elongated shape, to be delivered by percutaneous means without requiring the removal of any portion of the functional spine unit, in particular, keeping the nucleus and annulus intact. The shape, (including the thickness, height and/or length) of the device when in an expanded or inflated state has a larger profile such as illustrated in FIGS.3-11.

In certain embodiments of present invention, either during the implant procedure or in a subsequent procedure, the size or height of the implanted augmentation device may be selectively adjusted or varied. For example, after an initial assessment upon implant, it may be necessary to adjust, either reduce or increase, the size or height of the device to optimize the intended treatment.

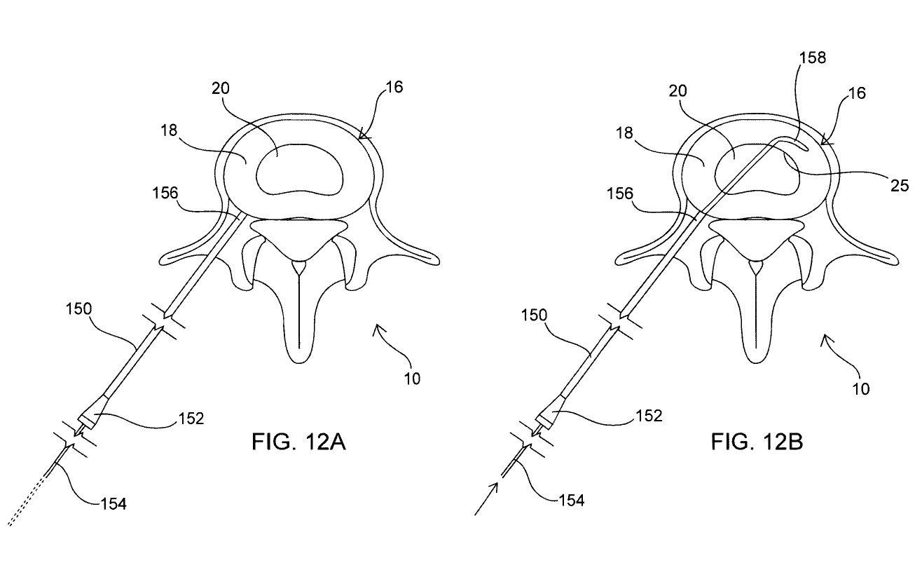

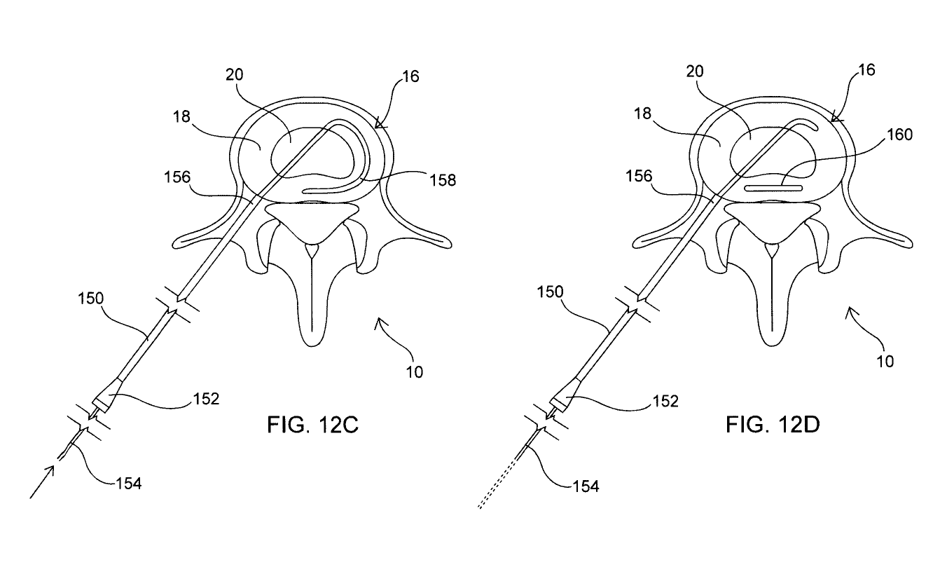

Methods of implanting the subject augmentation devices within an intra-annular space of an intervertebral disc are now described with reference to FIGS. 12A-12D. It should be understood that the procedure for implanting a single augmentation device of the present invention in the posterior portion of the annulus is shown for purposes of illustration and is not intended to be limiting with respect to the number of devices implanted and the location of the implant site.

To begin, a percutaenous puncture is made into the skin at a location lateral to the spinous process of the vertebra inferior to the intervertebral disc into which the augmentation device is to be implanted. As shown in FIG. 12A, the entry site is from the left side of the vertebral body 10, however, entry may be made from the right side or at any other accessible location depending on the intended location of the implant site. The puncture may be made by way of a sharp distal tip of a cannula 150 or by a trocar or stylet delivered through a lumen of cannula 150. Cannula or other tubular insertion tool 150 is advanced until its distal tip 156 is proximate the outer aspect of the posterior surface of annulus 18, as shown in FIG. 12A. A semi-flexible needle or a semi-rigid guide wire 154 is then inserted into hub 152 of cannula 150 and advanced therethrough and caused to penetrate into annulus 18. The extent of advancement of guide wire 154 through annulus 18 and through the disc is dependent upon whether the delivery path of the implant is to remain completely within the annular space or traverse the nucleus. If the delivery path is to remain completely within the annulus, the distal end 158 of guide wire 154 is advanced to the lamellar space into which the device is to be implanted and then deflected in a direction to minimize the amount of deflection necessary (i.e., in the illustrated view, to the right) and advanced circumferentially through the lamellar space (pathway not shown). If however, the delivery path is to include traversal of nucleus 20, distal tip 158 is penetrated through all of the lamellae (not individually illustrated for purposes of clarity) of the posterior portion of annulus 18 and advanced through and across the diameter of nucleus 20 and penetrated into an anterior portion of annulus 18, as shown in FIG. 12B. Alternatively, cannula 150 may be penetrated through the width of the nucleus up against an inner aspect 25 of an anterior portion of annulus 18 where after wire 154 is advanced through the cannula and need itself only penetrate the anterior portion of annulus 18. The former approach is preferable, however, in order to preserve the integrity of both the annulus and the nucleus.

Wire 154 preferably has a pre-curved configuration or is designed to be articulated whereby its distal end 158 is able to be passively steered or actively deflected within the annulus in a direction which is substantially transverse to its travel path thus far, i.e., in an anterior to posterior direction. In FIG. 12B, distal tip 158 is bent or deflected in a clockwise direction (although it may be selectively deflected in a counter-clockwise direction), having entered between adjacent lamellae (not shown) approximately within the central circumferential position of annulus 18. Distal tip 158 is further advanced in a clockwise direction within the annulus along a path which generally parallels the curvature of the annulus 18 until the target implant site within a posterior portion of the annulus has been reached, as shown in FIG. 12C. Finally, an implant 160 (schematically represented) is deployed from the delivery instrument(s) at the target implant site, as shown in FIG. 12D, and the wire 154 is retracted along the path it was delivered and removed from the body. If necessary, the procedure may be repeated for additional implants.

In certain embodiments, implant 160 is preloaded on the distal end of wire 154. Wire 154 may have an enlarged diameter just proximal of the loaded device in order to prevent the device from being pushed proximally along the wire while it is being delivered to the implant site. Alternatively, a catheter (not shown) may be advanced over wire 154 proximally of the implant so as to push the implant distally over wire 154 through the lumen of cannula 150 until it reaches the implant site. Still yet, implant 160 may be pre-loaded within the distal end of the catheter or advanced through the catheter after the catheter distal end has been positioned at the implant site. A pusher (not shown) advanced through the lumen of the catheter may be employed to advance the implant beyond the distal end of wire 154.

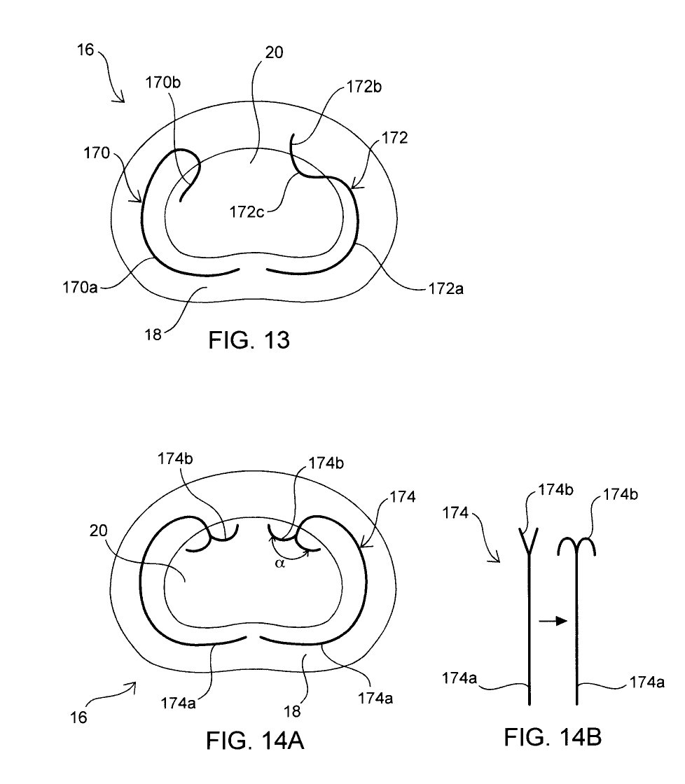

Referring now to FIGS. 13-18, there are illustrated various other devices of the present invention which are shown operatively implanted within an intervertebral disc 16.

In particular, each device has been partially implanted within the annulus 18 and partially implanted within the nucleus. Each figure illustrates two devices which have been implanted at opposite lateral sides of disc 16, however, such is not required nor intended to be limiting as only or more than two such devices may be implanted at any location within a single disc. Further, the devices may be two or more of a kind or may be different from each other, with their selection dependent upon certain factors such as the clinical need and physician preference.

As is evident from a review of the figures and the description below, for some of the devices embodiments, the majority of the device structure is positioned within the annulus and the remainder within the nucleus and, with other embodiments, about half of the device structure is positioned within the annulus and the other half is positioned within the nucleus. Of the latter embodiments, the extent to which the device structure is positioned within the annulus/nucleus may depend on one or more factors including, but not limited to, the length or size of the area of the annulus requiring augmentation, the extent to which the nucleus requires augmentation, the configuration and length of the device itself, and the clinical biomechanics of the particular disk being treated and its anticipated effects on the implanted device.

Devices 170 and 172 shown in FIG. 13 are made of a malleable material which enables their lengths to be bent to customized configurations. The desired configuration may be formed either prior to insertion into the disc or during insertion by way of delivery tools and techniques of insertion. As shown, a substantial portion of the lengths of both devices 170 and 172 has been positioned within the annulus 18 such as within an intra-lamellar plane, and having a radius of curvature substantially matching that of the annulus at that plane. One end 170a and 172a of each device has been positioned within a posterior portion of annulus 18 while the other end 170b and 172b has been bent and positioned so as to provide stability to the device and secure it from migration. In particular, end 170b has been bent or folded inwardly approximately 180° thereby crossing transversely through the lamellar layers situated between nucleus 20 and the layer in which device 170 is positioned, and into the nucleus where extends substantially parallel to the remainder of the device length.

End 172b of device 172 has also been bent inwardly to cross through the interim layers and into nucleus 20, but then has been curved in the opposite direction to enter back into annulus 18. As such only a relatively short distal portion 172c or device 172 resides within the nucleus. A particular annulus to be treated may have internal morphology that accepts varying degrees of intra- and extra-annular deployment of the device, making it more practical to position at least a portion of the augmentation device outside the annulus. Insertion of the distal end 172b into the annulus 18 provides for greater stability and decreases the likelihood of migration. Portion 170b of device 170 and portions 172b and 172c of device 172 provide a greater surface area of resistance to shear and compressive forces across the annulus.

FIGS. 14A and 14B illustrate a pair of devices 174 made of a superelastic material having a preformed biased configuration (shown) which, when operatively positioned within disc 18, reside within lateral portions, respectively, of disc 16 and symmetrically about nucleus 20. Further, the majority of the device lengths extend within an intra-lamellar space with one end 174a of each device situated within a portion of annulus 18 and the opposite ends 174b situated within nucleus 20. The latter ends 174b are each bifurcated, terminating in two branches which flare apart from each other by an angular distance α. This angle is typically in the range from about 45° to about 270°, but may smaller or larger if desired. As such, bifurcated end 174b serve to stabilize and prevent migration of each device 174. As with the remainder of the device, bifurcated ends 174b are made of a superelastic material which have a preformed flared configuration. Each of the bifurcate ends may be positioned relative to the annulus such that upon deployment, one or both of the flared ends extends back into the annulus, or visa-versa. Here, both flared ends have remained within nucleus 20. Each device 174 may be delivered in a restrained condition within a delivery catheter (not shown) and then be allowed to achieve a biased condition upon release from the catheter. Either end may be delivered as the leading end; however, one advantage in having bifurcated end 174b lead deployment (i.e., be deployed first) is that the device becomes immediately anchored.

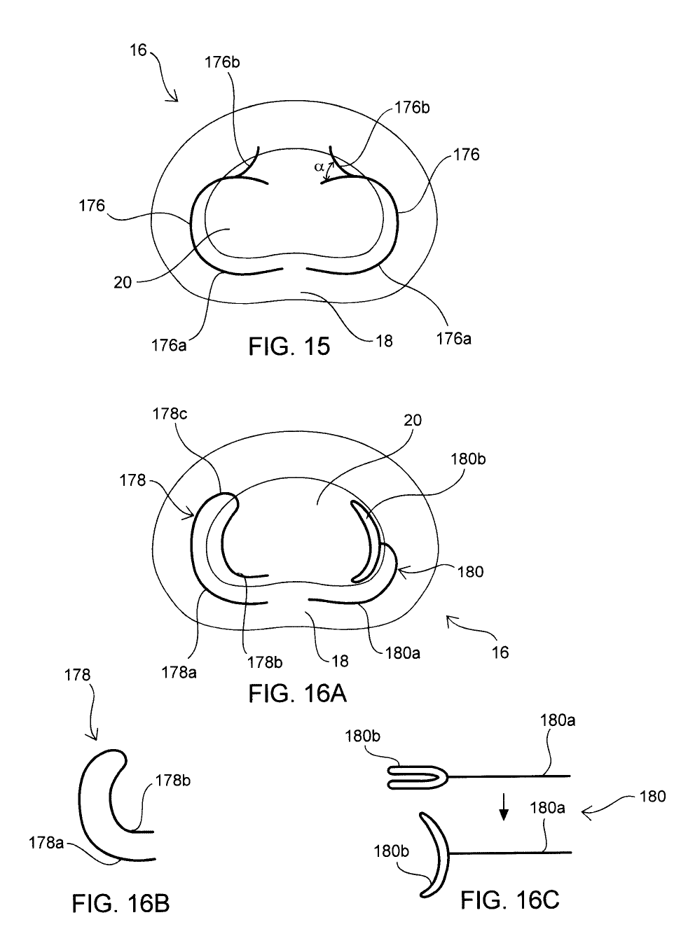

FIG. 15 illustrates another pair of augmentation devices 176 which are also made of a superelastic material and operatively implanted within disc 16 to be disposed symmetrically about nucleus 20. Device 176 has a configuration similar to that of device 174 of FIG. 14 with the difference being that the flared branches of bifurcated end 176b are biased to separate from each other by a smaller angle α. Additionally, ends 176b have been positioned relative to annulus 18 such that one of the flared branches extends back into the annulus.

FIGS. 16A-16C illustrate two other variations of augmentation devices of the present invention. Augmentation device 178 has curved U shape configuration in which the respective ends or legs 178a and 178b extend parallel and evenly with each other.

The legs have respective radii of curvature to match that of the portion of the disc in which they are to reside, i.e., leg 178a has a radius of curvature for placement in an intra-lamellar space and leg 178b has a radius of curvature for placement in the nucleus.

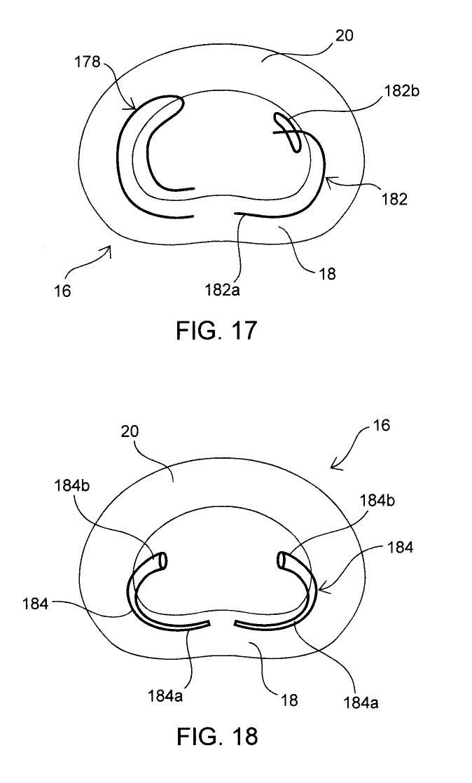

Augmentation device 180, as illustrated in 16C, comprises a stem 180a and an anchoring member 180b affixed at one end. Stem 180a is configured to be bent or otherwise conformed to fit within an intra-lamellar space and anchor 180b has a deployed configuration to extend perpendicularly to stem 180a and buttress against the inner wall of the annulus 18. As illustrated in FIG. 16C, anchor 180b has an undeployed or unbiased U shape with its legs folded distally and axially with stem 180a, so as to facilitate delivery to within disc 16. FIG. 17 illustrates another augmentation device 182 used in tandem with device 178 of FIGS. 16A-16C and similar to device 180. Here, anchoring member 182b is pivotally coupled to one end of stem 182a. As such, it may be axially aligned with stem 182a during delivery and then pivoted to a perpendicular position once positioned with the nucleus.

FIG. 18 illustrates another pair of augmentation devices 184, each positioned in a posterior-lateral portion of disc 16, respectively, where approximately half to two thirds of each device length resides within the annulus and the remaining half to third of the device length resides within the nucleus. Device 184 has a diameter or cross-sectional dimension which decreases or tapers along its length from end 184b to end 184a. The device may be pre-constructed with such a varying diameter or otherwise be made of or coated with a material that swells or expands upon implantation within an intervertebral disc 16. The thicker end 184b may serve the dual functions of anchoring the device to prevent migration and augmenting the nucleus. Conversely, the thinner end 184a allows positioning within a narrow intra-lamellar space.

The augmentation devices of FIGS. 13-18 may be delivered and deployed within an intervertebral disc in manners similar to those described above with respect to the augmentation devices of FIGS. 2-12.

The material composition and physical characteristics of a particular augmentation device will dictate, at least in part, the manner by which the device is deployed and/or expanded at the implant site. For example, for devices which are self-expanding upon release from a constrained position, e.g., expandable meshes, coils or strips which are wrapped or wound around the distal end of the guide wire, delivery through a catheter may be required. Alternatively, a very thin (possibly tear-away) sheath wrapped tightly around the device may be employed. Upon reaching the implant site, the sheath may be pulled off of and retracted proximally along the guide wire. The same may be used with hydrophilic implants which need to be protected from contacting fluids until positioned at the implant site. For augmentation devices requiring active deployment, a pull string or wire may be operatively attached to the implant and then actuated upon selective positioning of the device at the implant site. Balloon-type augmentation devices may be delivered in a flaccid or deflated condition, either exposed or covered (by a sheath or catheter) at the distal end of the guide wire, and coupled to an expansion or inflation medium such as by a tube or the catheter itself, if used. Upon positioning of the device, the medium is allowed to fill the balloon membrane to the desired capacity or until the implant achieves a necessary height.

It should also be noted that any of the above-described steps or procedures, including but not limited to cannulation of the target area, separation of the lamellae insertion of the subject implants within the target implant site, and the adjustment or readjustment of the implant may be facilitated by way of a scope delivered through a lumen of the delivery catheter and/or by way of various visualization techniques including but not limited to real time fluoroscopy, CT scanning or MR imaging, or a combination of preoperative CT or MR images superimposed onto a real time image tracking device, which are well known in the surgical arts.

One or more of the subject implants may be provided in a system which includes instrumentation, as described above, for delivering the implants to within a lamellar space within the annulus. Additionally, the subject devices and systems may be provided in the form of a kit which includes at least one augmentation device of the present invention. A plurality of such devices may be provided where the devices have the same or varying sizes and shapes and are made of the same or varying materials. The kits and/or systems may further include instruments and tools for implanting the subject devices, including but not limited to, a cannula, a trocar, a scope, a sheath, etc. The kits and/or systems may also include a supply of an inflation and/or expansion medium for the balloon-type augmentation devices. Instructions for implanting the subject systems and devices and for using the above-described instrumentation may also be provided with the kits.

It must be noted that as used herein and in the appended claims, the singular forms “a”, “an”, and “the” include plural referents unless the context clearly dictates otherwise. Thus, for example, reference to “a functional spine unit” may include a plurality of such functional spinal units and reference to “the catheter” includes reference to one or more catheters and equivalents thereof known to those skilled in the art, and so forth.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

That which is claimed is:

1. An intervertebral disc implant comprising:
a structure having a length, a first portion of which is configured for implantation within an intra-annular space of an intervertebral disc and a second portion of which is configured within a nucleus of the intervertebral disc, and having a height equal to or less than the normal height of the intervertebral disc into which it is to be implanted.

2. The intervertebral disc implant of claim 1, wherein the first portion and second portions have different lengths.

3. The intervertebral disc implant of claim 1, wherein the first and second portions have the same length.

4. The intervertebral disc implant of claim 1, wherein the first portion and second portions have different cross-sectional dimensions.

5. The intervertebral disc implant of claim 1, wherein the first and second portions have the same cross-sectional dimensions.

6. The intervertebral disc implant of claim 1, wherein the structure when implanted has a height in the range from about 3 mm to about 15 mm.

7. The intervertebral disc implant of claim 1, wherein the structure when implanted has a length in the range from about 5 mm to about 50 mm.

8. The intervertebral disc implant of claim 1, wherein the intra-annular space is between two adjacent lamellae.

9. The intervertebral disc implant of claim 1, wherein the structure has an expanded configuration and an unexpanded configuration.

10. The intervertebral disc implant of claim 9, wherein a cross-sectional dimension of the structure in the expanded configuration is about twice that of the unexpanded configuration.

11. The intervertebral disc implant of claim 9, wherein a cross-sectional dimension of the structure in the expanded configuration is about three times that of the unexpanded configuration.

12. The intervertebral disc implant of claim 9, wherein the structure is self-expanding upon implantation.

13. The intervertebral disc implant of claim 9, wherein the structure is actively expandable upon implantation.

14. The intervertebral disc implant of clam 1, further comprising an anchoring member at at least one end of the structure.

15. The intervertebral disc implant of clam 14, wherein the at least one end is bifurcated.

16. The intervertebral disc implant of clam 1, wherein at least the first portion of the structure has a construct and is made of materials that enable the implant to mimic the physical properties of a natural, healthy annulus of the intervertebral disc.

17. The intervertebral disc implant of clam 1, wherein the structure enables the natural motion of and supports normal physiological loads undergone by the intervertebral disc into which the device is implanted.

18. The intervertebral disc implant of clam 1, wherein the structure has physical characteristics which resist bending, fatigue failure, displacement, deformation, torsional stress, shear loads and other repetitive stresses and strains undergone by the intervertebral disc into which it is implanted during normal spinal motion.

19. The intervertebral disc implant of clam 1, wherein the height is greater than a thickness of the structure.

20. The intervertebral disc implant of claim 19, wherein the thickness-to-height ratio of the structure is no greater than about 1:3.

21. A method of treating an intervertebral disc having an annulus and a nucleus within an intervertebral space, the method comprising the steps of:
providing the implant of claim 1;
delivering the implant along a path which traverses the diameter of the nucleus; and
positioning the first portion of the implant in between two adjacent lamellae of the annulus.

22. The method of claim 21, wherein no portion of the intervertebral disc is removed.

23. The method of claim 21, wherein the first portion of the implant is positioned in a posterior or a posterior-lateral portion of the annulus.

24. The method of claim 21, wherein the implant upon implantation does not apply any compression to the annulus.

25. The method of claim 21, further comprising the steps of:
providing an insertion tool having a proximal end and a distal end;
inserting the insertion tool into the patient's back such the distal end is disposed at an outer aspect of the annulus or at an inner aspect of the annulus; and
wherein delivering the implant comprises utilizing the insertion tool.

26. The method of claim 21, wherein at least one portion of the implant is delivered in a low profile condition and further comprising expanding the at least one portion after being positioning the first portion in the annulus.

27. The method of claim 21, wherein the height of the intervertebral space is increased or maintained.

28. A system for augmenting the annulus of an intervertebral disc, the system comprising:
at least one implant according to claim 1; and
instrumentation for delivering the first portion to within a lamellar space within the annulus.

29. The system of claim 28, wherein the instrumentation comprises a guidewire having a deflectable tip.

30. A kit for augmenting the annulus of an intervertebral disc, the kit comprising:
at least one implant according to claim 1; and
instructions for implanting the at least one implant within the annulus.

31. The kit of claim 30, comprising a plurality of implants according to claim 1 wherein each implant has at least one of a thickness, a height and a length of the implants which varies from the other implants.
